(19) United States
(12) Patent Application Publication
(10) Pub. No.: US 2012/0015806 A1
(43) Pub. Date: Jan. 19, 2012

(54) NOVEL FORMULATION OF MICROBIAL CONSORTIUM BASED BIOINOCULANT FOR WIDE SPREAD USE IN AGRICULTURE PRACTICES

(76) Inventors: Sitaram Prasad Paikray, Delhi (IN); Vedpal Singh Malik, Fallschurch, VA (US)

(21) Appl. No.: 13/260,310

(22) PCT Filed: Mar. 25, 2010
(86) PCT No.: PCT/IB2010/051310

§ 371 Date: Sep. 25, 2011

(30) Foreign Application Priority Data
Mar. 25, 2009 (IN) 580/DEL/2009

(51) Int. Cl. A01N 63/04; C12N 1/20

(52) U.S. Cl. 504/117; 435/252.4

(57) ABSTRACT

The present invention relates to eco-friendly compositions and methods for providing plant growth enhancing formulations comprising mixtures of microbial isolates. The microbial consortium is developed for customized solution of soil health related problem such as with plant growth promoting properties including root and shoot length elongation, early and high germination rate, high yield, decrease in soil pathogenic load and increase soil micro and macronutrient status. These specifically designed polymicrobial formulations would further provide protection against plant pathogens lowering the need for nitrogen containing fertilizers, solubilize minerals, protect plants against pathogens, and make available to the plant valuable nutrients, such as phosphate, thus reducing and eliminating the need for using chemical fertilizers and chemical pesticides.

### FIELD OF THE INVENTION

The present invention relates to eco-friendly compositions and methods for providing plant growth enhancing formulations comprising mixtures of beneficial microbial isolates. Most particularly, microbial consortia comprised of bacteria, fungi and yeast. The invention further pertains to provide customized solution of soil health related problems balancing essential elements and other biomolecules improving soil health.

### BACKGROUND OF THE INVENTION

Plant rhizosphere contains billions of microorganisms in one gram of soil. These are either beneficial or neutral to plant growth. A number of microorganisms are known to be present in soil ecological niche (rhizosphere) having beneficial effects on plant growth. These beneficial plant growth promoting properties are nitrogen fixation, iron chelation, phosphate solubilization, inhibition of non-beneficial microorganisms, resistance to pest, can decompose plant material in soil to increase soil organic matter.

Pseudomonas is a ubiquitous microorganism and contains several plant growth promoting properties. The organism is known for the secretion of plant growth metabolites and auxins, producing compounds such as growth factors that directly increase plant growth. Moreover they also enhance plant growth by making unavailable micronutrient to plant by mobilizing them such as phosphate solubilization and iron chelation. Bacillus the second most dominant member of rhizosphere is also considered as plant growth promoting rhizobacteria. In addition to the above, nitrogen fixing bacteria are also well-known inhabitant of soil rhizosphere Likewise, plant materials decomposers are also naturally occur in soil but in low numbers. These bacteria increase the organic matter content in soil, which ultimately results in better crop improvement and plant productivity. Apart from the above-mentioned group of microorganisms, the effective microorganisms are also known to play important role in rejuvenation of plant and soil health. These can improve soil quality, plant growth, yield and quality of crops.

The success of a bioinoculant is dependent upon the survival of the microbial strain in the soil. The survival of the strain in adverse agro-climatic condition is very important and a big challenge. The best way to develop a bioinoculant is through the strains resistance to and able to survive under wide range of growth and storage conditions. The most important stress factors are high temperature, low temperature, acidity and alkalinity. So that a bioinoculant has to be developed using stress tolerant strains for its better survival under the field conditions. An organism is tolerant to either of one or two stress conditions naturally. However, the wide range of stresses is developed either by genetic manipulation or phenotypica adaptation. If a stress is developed by genetic manipulation using gene transfer technique, the strain will come under GMOs category, which is not considered safe to use in environment. But when, the adaptation to diverse conditions such as growth at high and low pH and temperature are developed by giving stress at gradually high or low variations so that the organisms develop stress at a particular condition phenotypically by adaptation instead of any genetic transfer. But the stress developed in this way is the stable phenotypic adaptation favoring the growth of organism under that condition. Such type of strain improvement is permanent and non-revertible.

Some beneficial organisms are effective in the laboratory only, but do not show their activity in the field, even after development of a product for market. Prior to the application, too little active material actually reaches to the field for application and rapid degradation occurs in the field. Formulation of a bioinoculant plays a vital role in helping to solve these problems and in making available critical numbers of organism for application in the field.

U.S. Pat. No. 5,697,186 discloses the use of microorganisms to enhance crop productivity and, more specifically, to the use of flocculated forms of bacteria, particularly Azospirillum and Rhizobium, or a combination thereof, as crop inoculants and delivery systems for other agriculturally beneficial microorganisms.

U.S. Pat. No. 4,551,164 discloses a composition of bacteria, specially Bacillus, and algae and methods for plant growth promotion. More particularly the invention concerns microbial plant growth promoting compositions and methods for their use.

U.S. Pat. No. 7,097,830 discloses synergistic bioinoculant composition comprising Bacillus strains isolated from cows, either individually or in all possible combinations, and optionally a carrier, with each of the strains showing plant growth-promoting activity.

U.S. Pat. No. 4,155,737 discloses to a process of inoculating microorganisms in plants in a polymer gel in which are embedded microorganisms. The invention is intended for controlling the productivity of cultivated plants.

(WO/2007/110686) application discloses a synergistic composition of at least one strain of Trichoderma harzianum or a combination thereof which is useful as bioinoculant.

Several microbial based bioproducts are commercially available and being used in the agriculture but the limitations of these products is in their composition and in their application to a particular crop. Most of the time, these products either contain only one plant growth property.

So for getting multiple benefits, the farmers have to apply best choice of products. Therefore, for better cropping practices it is desirable to develop a bio-product with multiple properties which can be used alone.

The formulation has been shown to enhance plant growth in a wide variety. Due to the novel microbial combination of Pseudomonas fluorescens, Pseudomonas striata, Bacillus polymyxa, Bacillus subtilis, Azospirillum, Rhizobium, Azotobacter, Trichoderma herzianum, Trichoderma viride, Saccharomyces cerevisiae and Lactobacillus and nutrients, the formulation of the invention provides an economical and effective alternative to conventional fertilizer intensive crop growing systems.

The patent describes the unique combination of these microorganism of action to provide a complementary, and occasionally synergistic benefit for plant growth, particularly under higher stress conditions such a nutrient deficiency, low moisture, and physical damage.

The present invention also shows comparison on a variety of plant types that the unique combination of selected both bacterial and fungal strains of the invention is effective in the enhancement of plant growth and health. Further, the present invention is directed to meet this agricultural demand.

It is an object of the present invention to overcome or at least alleviate one or more of the above-mentioned disadvantages of the prior art.

### OBJECT OF THE INVENTION

The main object of the present invention is to develop a high cell density novel formulation of microbial consortium of Pseudomonas fluorescens, Pseudomonas striata, Bacillus polymyxa, Bacillus subtilis, Azospirillum, Rhizobium, Azotobacter, Trichoderma herzianum and Trichoderma viride. The microbial consortium also contains effective microorganisms (EM) such as Saccharomyces cerevisiae and Lactobacillus.

Another object of the present invention relates to the use of microbial consortium as plant growth promoter.

Yet another object of the present invention relates to the use of microbial consortium as P-solubilizer, Nitrogen fixer, and plant residual matter decomposer, soil rejuvenator, soil and plant health enhancer.

Still another object of the present invention relates to the formulated composition to provide a high colony forming units (cfu) bacterial population with longer shelf life while maintaining the easy usability and handling of agriculturally important microbial bioinoculant.

Still another object of the present invention is to design a microbial consortium which is able to perform multidimensional activities in common.

### SUMMARY OF THE INVENTION

The present invention is directed to synergistic combinations (or mixtures) of microbial isolates. In addition, the present invention is directed to the microbial formulation to promote plant growth comprises a mixture of a bacteria fungi and yeast. The invention further pertains to a composition of selected potential strain of bacteria fungi and yeast.

Preferred Potential strains involves in the present invention viz Pseudomonas fluorescens, Pseudomonas striata, Bacillus polymyxa, Bacillus subtilis, Azospirillum, Rhizobium, Azotobacter, Trichoderma herzianum and Trichoderma viride, Saccharomyces cerevisiae and Lactobacillus.

The invention further pertains to the use of microorganisms in plant growth promotion, nutrient availability and in increasing soil organic matter content.

The invention also pertains to the above composition of mixed consortium developed herein is useful in wide application range which involves applying the mixture to plants, plant seeds or soil directly for getting effective results.

These specifically designed polymicrobial formulations would further provide protection against plant pathogens lowering the need for nitrogen containing fertilizers, solubilize minerals, protect plants against pathogens, and make available to the plant valuable nutrients, such as phosphate, thus reducing and eliminating the need for using chemical pesticides and chemical fertilizers.

Additionally, in present invention, wide application range refers broadly to improvements in yield of grain, fruit, flowers, or other plants harvested for various purposes, improvements in growth of plants parts, improved resistance to disease, improved survivability in extreme climate, and similar improvements of the growth and development of plants.

Significantly, these benefits to plants are obtained without any hazardous side effects to human, environments.

Further aspects of the invention will become apparent from consideration of the ensuing description of further embodiments of the invention. A person skilled in the art will realize that other embodiments of the invention are possible and that the details of the invention can be modified in a number of respects, all without departing from the inventive concept. Thus, the following descriptions are to be regarded as illustrative in nature and not restrictive.

### DETAILED DESCRIPTION OF THE INVENTION

These specifically designed polymicrobial formulations would provide protection against plant pathogens lowering the need for nitrogen containing fertilizers, solubilize minerals, protect plants against pathogens, and make available to the plant valuable nutrients, such as phosphate, thus reducing and eliminating the need of using chemical pesticides and chemical fertilizers.

The present invention is directed towards the isolation and screening of plant growth promoting microorganisms which includes Pseudomonas fluorescens, Pseudomonas striata, Bacillus polymyxa, Bacillus subtilis, Azospirillum, Rhizobium, Azotobacter, Trichoderma herzianum, Trichoderma viride, Saccharomyces cerevisiae and Lactobacillus.

The formulation has been shown to enhance plant growth in a wide variety. Due to the novel microbial combination of Pseudomonas fluorescens, Pseudomonas striata, Bacillus polymyxa, Bacillus subtilis, Azospirillum, Rhizobium, Azotobacter, Trichoderma herzianum, Trichoderma viride, Saccharomyces cerevisiae and Lactobacillus and nutrients, the formulation of the invention provides an economical and effective alternative to conventional fertilizer intensive growing systems.

The microorganisms in the present invention are useful in plant growth promotion, nutrient availability and in increasing soil organic matter content.

In an embodiment of the present invention, the said microbial consortium is provided in a composition suitable for treating plants or plant seed or directly to soil. The suitable carrier used in the invention is the powder. In this embodiment, several components present in the suitable carrier are growth supporting substances and the substances that maintains longer shelf life of the microorganisms present in consortium.

In another embodiment of the present invention, the composition contains the microbial cells in 10⁸-10⁹ CFU per gram of the carrier.

The present invention provides exemplary isolates of soil bacterial strains and fungal strains as described herein.

Specifically, the present invention provides an isolated Pseudomonas striata MTCC 5524 bacterial strain having accession number.

The present invention provides an isolated Pseudomonas fluorescens MTCC 5525 bacterial strain having accession number.

The present invention provides an isolated Bacillus subtilis MTCC 5527 bacterial strain having accession number.

The present invention provides an isolated Bacillus polymyxa MTCC 5528 bacterial strain having accession number.

The present invention provides an isolated Azospirillum brasilense MTCC 5526 bacterial strain having accession number.

The present invention provides an isolated Azotobacter sp. MTCC 5529 bacterial strain having accession number.

The present invention provides an isolated Trichoderma herzianum MTCC 5530 fungal strain having accession number.

The present invention provides an isolated Rhizobium sp. MTCC 5531 bacterial strain having accession number.

The present invention provides an isolated Trichoderma viride MTCC 5532 fungal strain having accession number.

The present invention provides an isolated Saccharomyces sp. MTCC 5533 yeast strain having accession number.

The present invention provides an isolated Lactobacillus bacterial strain having accession number.

The present invention provides an exemplary mixture of fungal isolates having accession number. The present invention provides exemplary microbial formulation, wherein said formulation consists of nitrogen fixing bacteria isolate, a phosphate solubilizing microbe isolate, a rhizobacteria isolate, and a biocontrol microbe isolate. In one embodiment said microbe is selected from the group consisting of a bacteria, fungus and yeast.

In one embodiment, said microbial formulation further comprises of a carrier, such that the microbial formulation of the present inventions are delivered to a seed or plant in a manner to promote growth and productivity, such as germination, yield, and the like. It is not meant to limit the type of carrier. Indeed, a variety of carriers are contemplated including but not limited to a liquid, a solid and a combination of a liquid and a solid carrier.

In particular for providing a benefit to a microbe or a plant, such as providing pathogen resistance, fungal resistance, reducing weeds, for example, an herbicide, a pesticide, a fungicide, a plant growth regulator, and for enhancing the effect of the microbial compound, for example, an encapsulation agent, a wetting agent, a dispersing agent, and the like. In one embodiment, said liquid carrier comprises water.

The present invention provides a method for enhancing plant growth, comprising of providing, i) a microbial formulation comprising a microbial soil isolate, wherein said microbial soil isolate is selected from the group consisting of bacterial strain, a Bacillus polymyxa MTCC 5528 bacterial strain having accession number, a Bacillus subtilis MTCC 5527 bacterial strain having accession number, an Azospirillum brasilense MTCC 5526 bacterial train having accession number, a Azotobacter sp MTCC 5529 bacterial strain having accession number, a Rhizobium sp. MTCC 5531 bacterial train having accession number, an Lactobacillus sp. bacterial strain having accession number, an Pseudomonas fluorescens MTCC 5525 bacterial strain having accession number, a Pseudomonas striata MTCC 5524 bacterial strain having accession number, a Trichoderma viride MTCC 5532 fungal strain having accession number, a Trichoderma herzianum MTCC 5530 fungal strain having accession number, a Saccharomyces sp MTCC 5533 yeast strain having accession number and ii) a plant, and applying said microbial formulation to a plant for enhancing plant productivity.

Further, an another embodiment of the present invention is directed to the microbial mixture of the isolates Pseudomonas fluorescens, Pseudomonas striata, Bacillus polymyxa, Bacillus subtilis, Azospirillum, Azotobacter, Rhizobium, Trichoderma herzianum and Trichoderma viride, Saccharomyces cerevisiae and Lactobacillus. which are useful in wide agriculture usages such as plant growth promoter, nutrient availability, and to improve soil and plant health. The formulation contains the organisms, which has a shelf life of two year with an initial CFU count of 10¹⁰ and at the end of one year not less than 10⁸.

As employed in this description, the term Bioinoculants refers to the population of single/multiple organisms present in a viable form, which increase plant growth and productivity. The formulation consists of mixed microbial population of live cells of Pseudomonas, Bacillus, Azospirillum, Azotobacter, Rhizobium, phosphate solubilizing bacteria, Trichoderma, Saccharomyces cerevisiae and Lactobacillus. The strains used herein were isolated from the rhizosphere and rhizoplane of the crops cultivated in Uttar Pradesh.

In accordance with another embodiment of the present invention, the strain selection was done by soil sampling from the rhizosphere of the crops grown in 300 clusters of Uttar Pradesh. A total of 300 samples from 300 different clusters were collected. Ten sampling was done from the same field and all the samples were pooled together to make a composite sample. Samples were processed immediately to recover maximum number of resident microflora on specific nutrient rich medium.

In accordance with still another embodiment of the present invention, Plant growth promotory activities were checked by siderophore production on chrome-azurol S plates, phosphate solubilization on phosphorous deficient medium containing tricalcium phosphate and auxins production.

Out of 1500 strains, 80% strains were found to be positive for siderophore production. Of which only 37% were strong (2.5-3.5 cm zone size) positive. Phosphate solubilization was shown by 40% strains. However, the combined data revealed that out of 1500 strains, ten strains contain all the tested properties. Of which, one Bacillus and one Pseudomonas strain was found potential growth promoting organisms.

In accordance with still another embodiment of the present invention, For the recovery of potent phosphate solubilizer, which is also the component of present invention, enrichment was performed in phosphate deficient medium. Direct soil samples from clusters of Uttar Pradesh were taken in liquid phosphate deficient medium and incubated the flask at 30° C., 200 rpm. After two to three successive transfers in the same liquid broth, plating was done and the clear halo zone was observed around the colonies.

The most potent phosphorus solubilizer was obtained with a zone size of 2.0 cm and 3.7 cm. The strain was selected as a potent phosphate solubilizing bacteria.

In vitro study on P solubilization was studied on both qualitative and quantitative scale. The solubilization was 35.67% quantitatively and 32 mm and 45 mm qualitatively in terms of zone formation. These organisms are Pseudomonas striata and Bacillus polymyxa.

In accordance with still another embodiment of the present invention, For the search of nitrogen fixers, sampling was performed from nitrogen deficient soil of marginal rain-fed region of U.P. The samples were enriched in Bromo Thymol Blue broth for the recovery of Azospirillum. A total of thirty soil samples were incubated for enrichment. The final bacterial strain was recovered after four successive transfers in the same broth and dilution plating on the bromo thymol blue medium.

The selection of free living aerobic nitrogen fixer, i.e., Azotobacter was done by enrichment technique in Jenson's broth followed by plating on same medium. The nitrogen fixing ability was measured by kjeldahl method.

In accordance with yet another embodiment of the current invention, the selection of potent Trichoderma was based on the bio-decomposition property of the organisms. The decomposition of plant material is a continuous process going on in the soil by the microbes. The increase in organic matter of soil in this way will reduce the side effect of chemical soil amendments and also improve the crop productivity and soil health.

In accordance with another aspect of the current invention, the plant growth promotory activity of the said strains was tested in soil by pot experimentation in green house. The organisms were applied by seed coating. The microbial culture was coated on seed by soaking and sown in the soils in the pot. Ten seeds per pot were sown and effect on seedlings growth was monitored. Ten replicates per organisms were maintained.

Increased percentage of seed germination results in improved crop growth and efficient seed use. Greater yield, increased grain size, and enhanced biomass production allow greater revenue generation from the given plot of land.

In accordance with another aspect of the current invention, the said organism was also applied directly to sterilized soil. 1 kg of soil was mixed with bacterial culture in a ratio so that per gram of soil contains 10⁸-10⁹ CFU. This was cross-tested by soil plating after mixing of culture with soil. The experiment was performed in green house and ten replicates per organisms.

In accordance with another aspect of the current invention, all the organisms were selected for stress tolerance. The stress factors included were acidity, alkalinity, high temperature and low temperature. These strains show growth profile under broad range of temperature (5° C. to 40° C.) and pH (4.0 to 8.0). The above said tolerance was induced in the organism of claim 1 through the process of induced stress tolerance wherein not all the organism was inherently tolerant to these stress conditions but was induced without any genetic manipulation.

The induction of a particular character in a microorganism by gradual developing stress at slightly altered condition will lead to the development of phenotypic adaptation that is stable and non-revertible. This type of organism modification/strain improvement will be non-dangerous to use and could not fall in the GMOs category.

In accordance with yet another aspect of the present invention, the formulation was designed to have high shelf life for which certain additives are added to increase shelf life of the microbes present in mixed consortium. The shelf life of the organism was studied at a wide range of temperature i.e. 5° C. to 40° C. and it was found that due to the addition of these formulants, they were able to with stand the temperature range and have a shelf life of 1 year wherein the initial cell density is 10¹⁰ and after the end of 1 year, it will not be less than 10⁸.

In accordance with yet another aspect of the present invention, the consortium is fermented for 2-3 days under present climatic condition in the presence of certain ingredients at farmers' field to enhance microbial counts and applied directly to the field in appropriate rate and timing to get proper response of the consortium. The fermenting material is the mixture of farm yard manure/organic manure/Agriculture waste, water, molasses/jaggary/sugar and/or besan/soybean floor.

Additionally, present invention, wide application range refers broadly to improvements in yield of grain, fruit, flowers, or other plants harvested for various purposes, improvements in growth of plants parts, improved resistance to disease, improved survivability in extreme climate, and similar improvements of the growth and development of plants.

In accordance with yet another aspect of the current invention, the product developed herein is applicable to wide range of crops including cereals (wheat and paddy), millets (maize, soybean and bajra), oilseeds (ground nut and mustard etc), pulses (chickpea, arhar, cowpea, blackgram, lentil and green gram etc), vegetables, fruits, spices and cucurbits.

In accordance with yet another embodiment of the present invention, field trial of the product in different blocks of Lucknow (Uttar Pradesh) has provided very important salient features of the culture developed, which as given in Annexure I.

Although the invention has been described with reference to specific embodiments, this description is not meant to be construed in a limiting sense. Various modifications of the disclosed embodiments, as well as alternate embodiments of the invention, will become apparent to persons skilled in the art upon reference to the description of the invention. It is therefore contemplated that such modifications can be made without departing from the spirit or scope of the present invention as defined.

1. A microbial formulation for plant growth with customized solution comprises at least seven beneficial bacteria; at least two beneficial fungi; at least one yeast; and at least one compound which extends the effective life time of said formulation.

2. The beneficial bacterial isolates as claimed in claim 1, herein are Pseudomonas fluorescens, Pseudomonas striata, Azospirillum, Azotobacter, Bacillus subtilis, Bacillus polymyxa, and Lactobacillus.

3. The beneficial fungal and yeast isolates as claimed in claim 1, herein are Trichoderma herzianum, Trichoderma viride and Saccharomyces cerevisiae respectively.

4. A microbial formulation as claimed in claim 1 is a synergistic composition useful as bioinoculant, wherein the said composition comprising at least one bacterial isolate of Pseudomonas striata, Pseudomonas fluorescens, Azospirillum, Bacillus subtilis, Bacillus polymyxa, Azotobacter, Trichoderma herzianum, Rhizobium sp. Trichoderma viride, Lactobacillus and Saccharomyces cerevisiae with an accession number MTCC 5524, MTCC 5525, MTCC 5526, MTCC 5527, MTCC 5528, MTCC 5529, MTCC 5530, MTCC 5531, MTCC 5532, MTCC 5523, respectively and optically carrier.

5. A microbial formulation, wherein said formulation consists of nitrogen fixing bacteria isolate, a phosphate solubilizing microbial isolate, a rhizobacterial isolate, and a biocontrol microbial isolate.

6. A microbial formulation effective for application to a plant or to soil which comprises of pseudomonas fluorescens, Pseudomonas striata, Azospirillum, Azotobacter, Bacillus subtilis, Bacillus polymyxa, Trichoderma herzianum, Trichoderma viride, Rhizobium sp., Lactobacillus and Saccharomyces cerevisiae.

7. A microbial synergistic formulation as claimed in claim 1, wherein the said composition has the ability of long shelf life.

8. A microbial formulation according to claim 1, wherein the microbial inoculant is effective for increasing plant productivity in legumes, non-legumes and vegetable crops.

9. A formulants optimized to achieve a shelf life of one year with an initial count of 10¹⁰ and after 1 year up to 10⁸ at a wide temperature range of 5° C.-40° C.

10. The microbial formulation of claim 4, wherein the said carrier is powder.

11. The microbial formulation of claim 4, wherein the said powder carrier comprises of talcum and/or Aluminum silicate and/or a mixture thereof.

12. The formulants optimized as claimed in as in claim 9, wherein the said formulants added are polyvinyl pyrollidone and polyethylene glycol.

13. The microbial formulation of claim 10, further comprising, a liquid carrier.

14. The microbial composition according to claim 6, wherein the composition improves phosphorous solubilization in soil.

15. The microbial composition according to claim 6, wherein the composition has the ability to promote plant growth.

16. The microbial composition according to claim 6, wherein the composition improves nitrogen fixation in free living environment.

17. The microbial composition according to claim 6, wherein the composition improves nitrogen fixation in microaerophilic environment.

18. The microbial composition according to claim 6, wherein the composition improves soil rejuvenator.

19. The microbial composition according to claim 6, wherein the composition improves is nutrient cycling.

20. The microbial composition according to claim 6, wherein the composition improves is partly to organic matter decomposition.

21. A microbial synergistic formulation as claimed in claim 1, wherein the said composition has the ability to promote plant growth.

22. A microbial synergistic formulation as claimed in claim 1, wherein the said composition has the ability to tolerate abiotic stresses.

23. A microbial synergistic formulation as claimed in claim 1, wherein the said composition has the ability to induce systemic resistance in plants.

24. A method imparting to soil microbial consortium as in claim 1 comprising application to direct soil before sowing, soil surrounding plants and as seed treatment.

25. A method for enhancing plant growth, comprising, a) providing, i) A microbial formulation, wherein said formulation comprises a mixture selected from the group consisting of a bacterial mixture having accession number and a fungal mixture having accession number, and ii) a plant, and b) applying said microbial formulation to a plant for enhancing plant productivity.

26. The method imparting to the consortium application comprising on-site enrichment and multiplication of microbial population.

27. The multiplication of microbial population as claimed in claim 26, wherein the multiplying agent is agriculture waste/organic manure/farm yard manure and glucose/jaggary/molasses.

28. The method as claimed in claim 24, wherein the said microbial formulation is applied to soil to provide 10⁶ to 10⁸ cfu/g of soil.

29. The method as claimed in claim 24, wherein the said microbial formulation is applied to seed to provide 10⁷ to 10⁹ cfu/g of seed.
